(19) United States
(12) Patent Application Publication
(10) Pub. No.: US 2007/0020321 A1
(43) Pub. Date: Jan. 25, 2007

(54) Method for enhancing attenuation characteristic of absorbent materials useful with dermal and transdermal substance delivery systems

(76) Inventors: Bruce K. Redding, Broomal, PA (US); Ram B. Roy, Collegeville, PA (US)

Correspondence Address: PLEVY & HOWARD, P.C., P.O. BOX 226, FORT WASHINGTON, PA, 19034, US

(21) Appl. No.: 11/361,699

(22) Filed: Feb. 24, 2006

Related U.S. Application Data
(63) Continuation-in-part of application No. 11/130,817, filed on May 17, 2005.
Application No. 11/130,817 is a continuation of application No. 10/345,825, filed on Jan. 16, 2003, now Pat. No. 6,908,448.
(60) Provisional application No. 60/655,362, filed on Feb. 24, 2005.

(51) Int. Cl. A61L 15/16

(52) U.S. Cl. 424/447

(57) ABSTRACT

A method for preparing a patch containing at least one substance to be delivered into a user, the method including: providing an absorbent material; introducing the at least one substance into the material; placing the material inside the patch; and, sealing the substance holding material containing patch. The absorbent material may be insonified. The absorbent material may be freeze dried. The absorbent material may be chemically treated. The absorbent material may be cross-linked with the substance.

### RELATED APPLICATIONS

This application claims priority of U.S. Patent Application Ser. No. 60/655,362, entitled METHOD FOR THE ATTENUATION ENAHANCEMENT OF ABSORBENT MATERIALS USED IN ULTRASONIC TRANSDERMAL DRUG DELIVERY SYSTEMS, filed Feb. 24, 2005, and is a continuation-in-part of U.S. patent application Ser. No. 11/130,817, filed May 17, 2005, entitled SUBSTANCE DELIVERY DEVICE, which is a continuation of U.S. patent application Ser. No. 10/345,825, filed Jan. 16, 2003, entitled SUBSTANCE DELIVERY DEVICE, now U.S. Pat. No. 6,908,448, the entire disclosures of each of which are hereby incorporated by reference as if being set forth in their respective entireties herein.

### FIELD OF THE INVENTION

The present invention relates generally to dermal and transdermal substance delivery systems, and particularly to facilitating the liberation of substances to be delivered from absorbent materials incorporated in substance delivery patches.

### BACKGROUND OF THE INVENTION

Generally, transdermal medicinal compound, or drug, delivery systems employ a medicated patch, which is affixed to the skin of a patient. The patch allows a medicinal compound contained within the patch to be absorbed through the skin layers and into the patient's blood stream. Transdermal drug delivery generally mitigates the pain associated with drug injections and intravenous drug administration, as well as the risk of infection associated with these techniques. Transdermal drug delivery also avoids gastrointestinal metabolism of administered drugs, reduces the elimination of drugs by the liver, and provides a sustained release of the administered drug. Transdermal drug delivery also enhances patient compliance with a drug regimen, because of the relative ease of administration and the sustained release of the drug.

However, many drugs are not well suited for conventional administration via known transdermal drug delivery systems since they are absorbed with difficulty through the skin due to the molecular size of the drug or to other bio-adhesion properties of the drug. In these cases, when transdermal drug delivery is attempted, the drug may pool on the outer surface of the skin, not permeating through the skin into the blood stream. An example of a drug exhibiting such a characteristic is insulin, which has been found difficult to administer via conventional transdermal drug delivery systems.

Accordingly, these types of medicinal compounds, or medications or drugs, are often administered either by injection or oral dosage forms, which each present certain drawbacks. Increased dosages are sometimes necessary. For example, chemotherapeutic agents are conventionally administered in increased dosages because of their need to survive degradation in the gastrointestinal tract. Further, many critical treatments for AIDS require a cocktail of drugs taken orally in solid dosage forms, several times a day to be effective. While it may be desirable to deliver medicinal compounds transdermally, these medications are not suitable for administration via conventional transdermal drug delivery systems because of the extensive dosing requirement, as well as the inability of the drug molecule to remain stable in a form suitable for transdermal delivery. Applicants believe the unsuitability of many drugs for conventional transdermal delivery results, at least in part, from low bio-absorbance of the drug across the skin layers.

Accordingly, conventional transdermal drug delivery methods have been found suitable only for low molecular weight medications—such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with conventional transdermal drug delivery methods.

There are three basic designs to transdermal patches: a reservoir type patch, a matrix type patch and a Drug In Adhesive (DIA) type patch. FIG. 1 schematically illustrates a reservoir type transdermal patch construction 100. Reservoir patch 100 includes a liquid reservoir compartment 110 containing a substance 120, such as a drug solution or suspension, which is separated from a release liner 130 by a semi permeable membrane 140 and an adhesive 150. A backing layer or support 160 is also typically provided. Commercially available examples of reservoir type patches include Duralgesic® (Fentanyl), Estraderm® (estradiol), and Transderm-Nitro® (Nitroglycerin).

FIG. 2 illustrates a matrix type patch 200. Similar to the reservoir patch 100 shown in FIG. 1, patch 200 includes a release liner 130, adhesive 150 and backing layer 160. Distinctly, the drug is provided within semisolid formulation 220 though, and there is no membrane layer. Commercially available examples of matrix type patches include Habitol® (Nicotine), and Nitrodisc® (Nitroglycerine and ProStep® (Nicotine).

A DIA type patch is generally characterized by the drug being incorporated within, or in direct contact with, a skin-contacting adhesive. In such a configuration, the adhesive fulfills the adhesion-to-skin function and serves as the formulation foundation, containing the drug and excipients. DIA patches may generally be monolithic or multilaminate in nature. Patch 200 of FIG. 2 can also be characterized as a multilaminate DIA matrix patch construction. Commercially available DIA patches include Climara® (Estradiol), which is monolithic, and Nicoderm® (Nicotine), which is multilaminate.

A DIA patch design provides advantages in reducing the size of the overall patch and providing a good concentric seal upon the skin. DIA patches also tend to be comfortable to wear as compared to reservoir and matrix patches. A typical DIA patch is 165 to 200 μm thick. Disadvantages of DIA patches include a longer drug delivery profile—as the release of the drug from a DIA patch follows first order kinetics, i.e., is proportional to the concentration of drug within the adhesive. As the drug is delivered from the DIA patch, drug concentration falls such that the delivery rate falls over time.

Regardless of patch type, intermingling of the drug with adhesive compositions represents a problem—as new drug profiles result and the drug may tend to degrade through the interaction with the adhesive composition. For example, the chemistry of the adhesive can alter the stability, performance and/or function of certain drugs. Additionally there are limits to the molecule size of drugs, which can be delivered via a passive system. Typical drug candidates are below 500 Daltons for DIA patches and below 1,000 Daltons for matrix and reservoir patches, even with the use of skin enhancers.

Electric Potential (lontophoresis) and ultrasound (phonophoresis) have been proposed to enhance transdermal delivery, e.g. assist delivery. These systems are typically designed to either increase the flow of metallic based drugs across the stratum corneum, micro-porate the skin or allow the delivery of macromolecules across the stratum corneum into the dermis or underlying tissue. Such assisted transdermal delivery devices (TDD's) typically use an external electronic system, separate from a drug-containing patch. The patch may typically incorporate electrodes that assist with ionic transfer.

Improved methods and devices for facilitating transdermal delivery of drugs are desirable.

### SUMMARY OF THE INVENTION

A method for preparing a patch containing at least one substance to be delivered into a user, the method including: providing an absorbent material; introducing the at least one substance into the material; placing the material inside the patch; and, sealing the substance holding material containing patch. The absorbent material may be insonified. The absorbent material may be freeze dried. The absorbent material may be chemically treated. The absorbent material may be cross-linked with the substance.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts and in which:

FIG. 1 illustrates a reservoir type patch;

FIG. 2 illustrates a DIA matrix type patch;

FIG. 3 illustrates an absorbent pad incorporating patch;

FIG. 4 illustrates the networks of various materials possessing suitable for absorption properties; and,

FIG. 5 illustrates a Franz Diffusion cell used to test certain principles of the present invention.

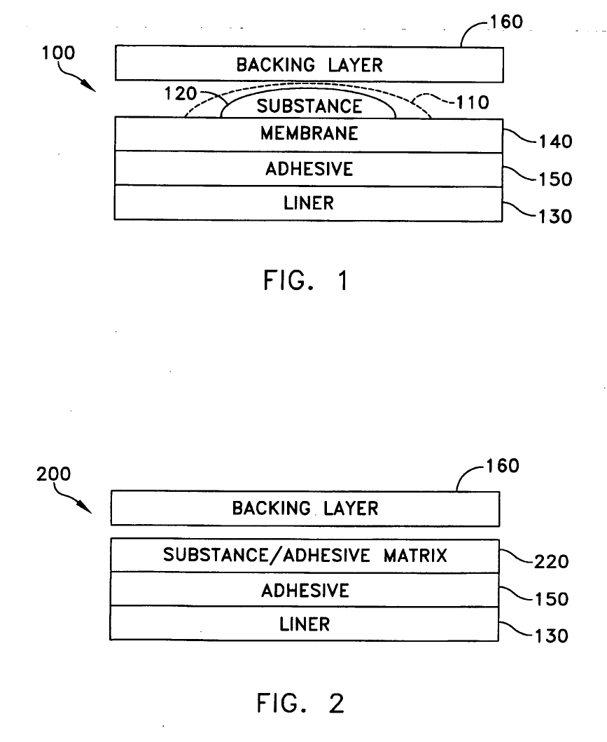

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in typical patches and dermal and transdermal delivery assisting methods and systems. However, because such elements are well known in the art, and because they do not facilitate a better understanding of the present invention, a discussion of such elements is not provided herein. The disclosure herein is directed to all such variations and modifications known to those skilled in the art.

Preliminarily, it should be understood that while the preferred embodiments of the present invention are discussed as they relate to transdermal drug delivery, they have a broad applicability covering active/passive dermal and transdermal delivery of drugs and other substances. For example, dermal delivery of non-medicated skin creams may also be improved using the principles of the present invention.

To solve the problem of drug contamination afforded by those systems employing adhesive matrix designs, wherein drug contamination or denaturing may result due to the interaction with an adhesive or polymer component, the parent application hereof, now U.S. Pat. No. 6,908,448 proposes using an absorbent pad. The absorbent pad acts to absorb the drug. The absorbency power of a pad is typically measured in factors of liquid water absorption. For example, many absorbent materials can hold up to twelve times their weight in liquid. Hence an absorbent pad can typically contain far more liquid suspension than solid composition of a particular drug.

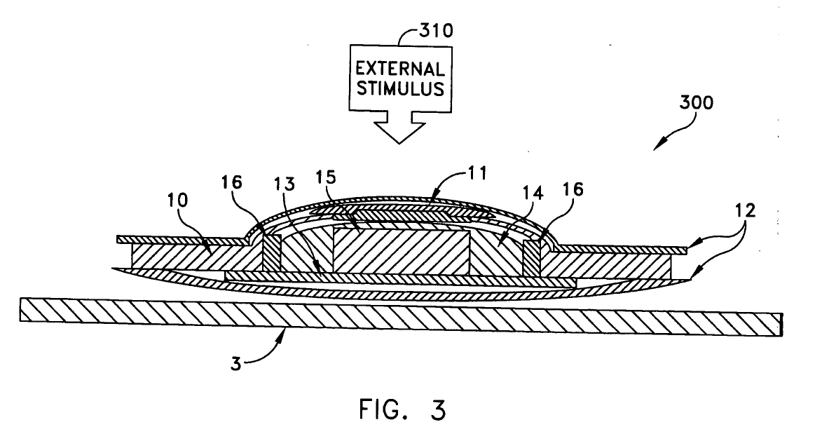

FIG. 3 illustrates a transdermal patch 300 suitable for use with ultrasonic signals, wherein the patch employs an absorbent pad. Patch 300 is constructed with a backbone or backing material 10 into which a section, or aperture, has been created. In the illustrated embodiment the aperture accommodates a sonic membrane 11. A peel-away film 12 seals patch 300 until use. Peel-away film 12 may be constructed of any suitable material, including, but not limited to, UV-resistant, anti-static polyethylene film (50 micrometer thickness) available from Crystal-X Corp., of Sharon Hill, Pa. In the illustrated embodiment, and oppositely disposed from membrane 11, is a semi-permeable member 13. Member 13 may take the form of a membrane or film that comes into functional proximity with the skin of a user. For example, the patch may be adhered to the skin such that membrane 11 is in direct contact with the skin. In the interior of patch 300 is an absorbent pad 14 that holds the desired drug or medicinal compound 15. In the illustrated embodiment a gasket 16 is placed between backbone 10 and absorbent pad 14. Gasket 16 may be composed of any suitable material, such as, for example, synthetic rubber. Gasket 16 forms a reservoir or well over which absorbent pad 14 is placed. When pressed upon the skin, gasket 16 forms a barrier, which tends to restrict moisture and air from traveling under the patch and interfering with the ultrasonic signal intensity. Alternatively, a sealant compound, ultrasonic gel or other suitable material may be used for or in place of the gasket 16 to provide a sealing action around the borders of patch 300 to provide moisture protection, prevent leakage of substance or the drug from the patch and prevent air from entering under the patch. Of course, numerous changes to the components or construction of patch 300 may be made without departing from the spirit of the invention disclosed herein.

Regardless, an external stimulus, such as a source of ultrasonic signals, transmits signals 310 into patch 300, and pad 14, through sonic membrane 11. Drug or other substance 15, contained within the absorbent pad 14, is released in response to the impinging ultrasonic signals. The substance then passes through semi-permeable membrane 13 and is deposited on, in or through the surface of the patient's skin 3. While patch 300 has been described for use with ultrasound 310, other forms of external stimulus may be used in addition or in lieu of ultrasound. For example, iontophoresis, heat therapy, radio waves, magnetic transmission lasers, microwave signals, and/or electric currents applied to the skin may be used as the external stimulus. For example, ultrasonic signals may be used together with iontophoresis, or ultrasound may be used as a pre-treatment to the application of iontophoresis.

Adhesives that would otherwise directly contact the drug 15 are eliminated in patch 300. Adhesives may be used in the border of the patch, but the DIA, matrix or reservoir designs are discarded in favor of incorporating absorbent pad 14, which is held within the transdermal patch.

Further, in the reservoir, matrix and DIA types of transdermal patches there is typically a low concentration of drug—such that delivery is typically dependent upon the surface area of the patch. In an absorbent pad including configuration though, the thickness of the absorbent pad can be varied to leverage the pad material absorbency factor, so that more of a drug can be contained within an absorbent pad of particular lateral dimensions. For example 1 sq. cm of cellulostic pad can hold up to 12 times its weight in moisture at 1 mm thickness. A nylon pad of the same thickness may hold only 3 times its weight in moisture. By varying the material used and altering the thickness, the absorbent pad's holding capacity can be adjusted to meet a desired release rate and longevity, exceeding that of conventional reservoir, matrix or DIA patches.

Accordingly, incorporating an absorbent pad addresses critical concerns limiting the use of transdermal patches in drug delivery applications, especially when used in combination with assisted delivery methods.

It has been found, however, that the absorbent pad material may have an affinity for a drug to be delivered. If the absorbent material is nylon for example, insulin may adhere to the nylon fibers, such that it is not readily liberated from the patch in either a passive or assisted (e.g., active) transdermal delivery device (TDD) configuraton. For non-limiting purposes of explanation, a passive TDD, which takes the form of a transdermal patch, delivers a drug from the patch to the surface of the skin where the compound is then absorbed into the dermis. Air pockets, moisture and impurities within the absorbent material all tend to deleteriously affect delivery. Some drugs may tend to adhere to the fibers of the absorbent material and not liberate from the patch in an even delivery distribution rate. The impurities within the absorbent material can interact with the drug and contaminate the compound. Air pockets, moisture and surface tension within the fiber material may tend to retard the drug delivery rate from the absorbent material. Even a passive form of transdermal patch, which employs an absorbent pad type of construction can be limited in its scope of delivery by interaction between the drug and the material used to form the absorbent pad. It is desirable to therefore address these potential shortcomings of absorbent pad including patches.

According to an aspect of the present invention, drug release characteristics of an absorbent pad incorporating patch are improved by pre-treating the absorbent material prior to drug introduction, such as prior to being incorporated within the patch. For example, one or more ultrasound signals may be applied to the absorbent pad material. This may tend to drive air pockets from the pad material and change the surface tension of the material, and reduce the material affinity for one or more drugs. As a further example, the absorbent material may be freeze dried. This may tend to drive impurities from the material and alter the surface tension, and reduce the material's affinity for one or more drugs. As another example, the absorbent material may be washed. This may also tend to drive impurities from the material and alter the surface tension, to reduce the material's affinity for one or more drugs.

Pre-treatment is suitable for use with a great number of absorbent materials useful in patches. Nonetheless, materials particularly well suited to be processed in this manner and incorporated into a patch may possess one or more of the following characteristics. They may provide for a high absorbency for a selected drug presented in an emulsion or solution form. They may be inert with respect to the select drug, or its excipient or preservatives used in the solution form of the drug, over a protracted period of storage time. They may be resistant to degradation under exposure to a delivery assisting source, e.g., ultrasonic transducer(s), and to releasing contaminants into the stored drug. They may be essentially free of metallic, organic or inorganic contaminants. They may be non-irritating to human skin and remain stable upon interaction with human sweat. They may remain stable in a stored form for one year or more and be resistant to degradation with time when soaked with the drug. They may be composed of natural and/or synthetic materials. They may be capable of absorbing about fourteen (14) or more times their weight in liquid (e.g., be superabsorbent).

Such a superabsorbent material provides the pad with the capacity to store the drug in a dilute solution or suspension. This may prove particularly advantageous for polypeptides, such as insulin, which is believed to form multimeric structures when concentrated in solution. Preventing the absorbent pad from drying out, and thus maintaining insulin in dilute solution, maintains the insulin in monomeric form, which is most easily transported out of the patch and through the skin.

Further, when used with an assisting configuration, e.g., active delivery system, the absorbent material may contain functional groups that cross-link with the drug. Such cross-linking may act to stabilize the drug for storage while in the patch. When an ultrasonic signal is applied through the patch, it disrupts the cross-linking—thereby releasing the drug from the absorbent pad and freeing the drug for delivery to the subject. The absorbent material may contain a moderate number of cross-linking points, such that the absorbent material forms cross-linkages with the drug, but does not form cross-linkages that disrupt the native structure of the drug, and such that, upon exposure to assisting signals, e.g., ultrasonic signals, releases the cross-linking such that the drug is no longer bound to the absorbent pad and is free to be delivered to the subject.

In one embodiment, the absorbent material and drug are cross-linked through hydrogen bonding. In such a case, the absorbent material contains functional groups that form hydrogen bonds with functional groups of a polypeptide drug, such as, for example, insulin. The hydrogen bonding acts to stabilize the structure of the drug. Upon exposure to ultrasonic signals, the hydrogen bonding that cross-links the drug to the absorbent material is disrupted without breaking the hydrogen bonds that form the native secondary structure or other aspects of the structure of the polypeptide.

The following are particular materials that may be utilized in the construction of an absorbent pad, all by way of non-limiting example: cellulose fiber pads, cotton, natural sponge, woven cloth fabrics, polyurethane foams, polyisocynurate foams, non-woven cloths, fused silica, starch, corn meal, wood pulp fibers, collagen pads, poly methyl methacrylate, polyvinyl alcohol, poly vinyl pyrrolidine, poly acrylic acid, poly (2-hydroxy ethyl methacrylate, polyacrylamide, poly ethylene glycol, polylactides(pla), polyglycolides(pga), poly(lactide-co-glycolides), polycarbonate, chitosan, poly (n-isopropylacrylamide), co-polymer formulations of poly methacrylic acid and poly ethylene glycol, co-polymer formulations of poly acrylic acid and poly (n-isopropylacrylamide), hyrdogels, e.g. polyacrylamide, poly(propylene oxide, pluronic polyols family of gel materials, e.g. pluronic-chitosan hydrogels, and silica gels. Other natural or synthetic materials, which absorb the drug compound and release the drug upon excitation, are also suitable for use.

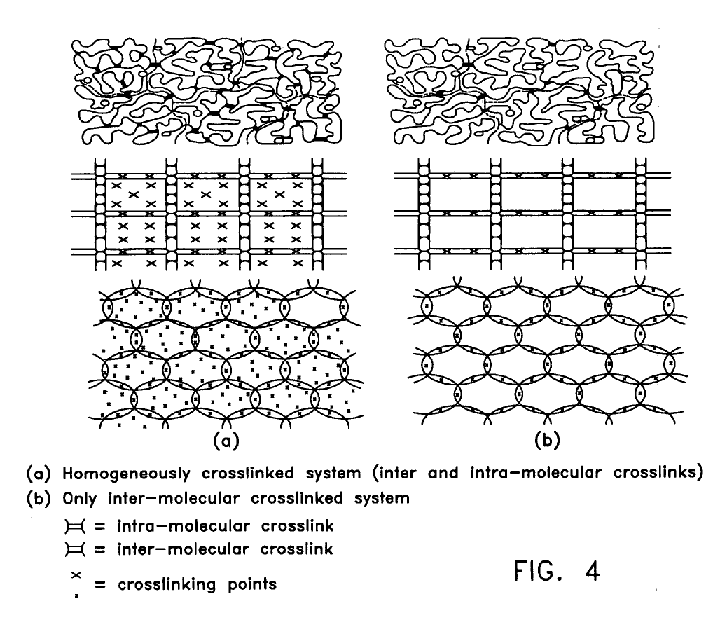

FIG. 4 illustrates the network of various materials possessing suitable absorption properties. FIG. 4 illustrates that the absorbent material may possess several weave patterns which aid in its absorbency. Each weave pattern in itself may tend to enhance or retard the delivery of a stored drug. By pre-treating the pad material, the hold on the drug by the material, or by the weave pattern, can be loosened.

Such absorbent materials are useful for delivering a wide variety of substances to a patient. The substances may be delivered, for example, dermally, transdermally, transcutaneously, intralumenally, and within solid tissue sites. The absorption of the substance or a pharmacologically active portion thereof into the underlying or surrounding tissue may be phonophoretically assisted by the application of ultrasonic or sonic energy to an absorbent pad containing patch. The substance may take any suitable form, including, but not limited to, liquids, gels, porous reservoirs, inserts, or the like, and the substance or pharmacologically active portion thereof may, for example, treat or alleviate an existing condition or prophylactically prevent or inhibit another condition of the patient. The effect of the substance may be local, such as providing for anti-tumor treatment, or may be systemic. Suitable medicaments include, but are not limited to, broad classes of compounds normally delivered through the skin and other body surfaces or into solid tissues.

In general, and by way of non-limiting example, suitable medications include or incorporate: anti-infectives such as antibiotics and antiviral agents; analgesics and analgesic combinations; anorexics; anti-helminthics; anti-arthritics; anti-asthmatic agents; anticonvulsants; antidepressants; anti-diabetic agents; anti-diarrheals; antihistamines; anti-inflammatory agents; anti-migraine preparations; anti-nauseants; anti-neoplastics; anti-parkinsonism drugs; anti-pruritics; anti-psychotics; antipyretics; antispasmodics; anti-cholinergics; sympathomimatics; xanthine derivatives; cardiovascular preparations including, but not limited to, potassium and calcium channel blockers, beta-blockers, and anti-arrhythmics; anti-hypertensives; diuretics; vasodilators including general coronary, peripheral and cerebral; central nervous system stimulants; cough and cold preparations, including decongestants; hormones, including, but not limited to steroids, including, without limitation, estradiol, and corticosteroids; hypnotics; immunosuppressives; muscle relaxants; parasympatholytics; psychostimulants; sedatives; and tranquilizers. Ionized and non-ionized drugs may be delivered, as can drugs of high or low molecular weight.

Proteinaceous and polypeptide drugs represent one class of drugs suitable for use in conjunction with the presently disclosed invention. Such drugs cannot generally be administered orally in that they are often destroyed in the gastrointestinal tract or metabolized in the liver. Further, due to the high molecular weight of most polypeptide drugs, conventional transdermal delivery systems are not generally effective. Examples of pharmaceutical or nutritional compounds that may be contained within an absorbent pad containing patch include, but are not limited to: acetaminophen, aspirin, corticosterone, erythromycin, ibuprofen, insulin, nitroglycerin, nicotine, steroids, including without limitation, progesterones, estrogens, for example, estradiol, and vitamins. Suitable forms of insulin include, but are not limited to, Humulin® and Humulog®, both available from Eli Lilly and Company, Indianapolis, Ind. Any other substance, including, but not limited to, pharmaceutical and/or nutritional compounds used for nutraceutical, medicinal or pharmaceutical purposes, and combinations thereof, may also be utilized. It may also desirable to use the method of the invention in conjunction with drugs to which the permeability of the skin is relatively low, or which give rise to a long lag-time.

In one embodiment of the invention, the absorbent material is pre-treated by freezing, followed by vacuum drying. Such freeze-drying of the absorbent material may reduce the amount of contaminants, such as air or moisture otherwise trapped in the absorbent material. Such contaminants may otherwise react with functional groups of the absorbent material, thus preventing these functional groups from forming cross-links with the drug. Upon freeze-drying, the contaminants are removed, thus freeing the cross-linking sites of the absorbent material such that the sites are free to form cross-linkages with the substance to be delivered. In addition, the freeze-drying may remove contaminants that otherwise might react with or contaminate the drug.

In another embodiment, the absorbent material is pre-treated by washing with alcohol or another solvent, and then freeze-dried. This pre-treatment may tend to drive moisture and air from the absorbent material and improve the materials sonic resonance characteristics, while also removing contaminants. This procedure is also conducive to altering the surface tension of the absorbent material, in particular the fiber strands, to the point where a particular substance within the material is more easily liberated via excitation, e.g., ultrasound. By way of further explanation, the absorbent pad material may be pre-treated by being soaked in an aqueous solution of 0.9% NaCl. Thereafter, the pad may optionally be freeze-dried. The pre-treatment with the saline solution provides a residue of NaCl that remains in the absorbent material. The salt residue acts as a humectant, attracting water and thus maintaining some moisture within the absorbent pad. This prevents the absorbent pad from drying out and allows a drug stored in the pad to remain in solution, preventing loss of moisture that may cause the drug solution to become increasingly concentrated. Concentration of the drug solution may lead to aggregation or precipitation of the active drug from the solution, impeding drug transport.

In another embodiment, the absorbent material is pre-treated using ultrasound. The pre-treating ultrasound may be in the range of frequency and intensity intended to be used to liberate the drug from the pad. For example, 75 units of insulin stored within a cellulose pad may be liberated with an about 20-150 KHz ultrasound signal having a 125 mw/cm² intensity. Other frequencies, intensities, and/or combinations thereof may be used though. Regardless, the cellulose pad is treated at around the same ultrasonic frequency and intensity level as the pre-treating signaling. This pre-treatment may tend to drive moisture and air from the absorbent material and improve the homogenous nature of the pad and material's sonic resonance characteristics.

The following materials were selected for attenuation enhancement experimentation.

### Material # 1

Material: Cellulose Pad, Wood Pulp with ethylene vinyl acetate based synthetic latex. supplier Model No.: Vicell # 6009, supplied by: Buckeye Absorbent Products, 1001 Tillman St., PO box 80407, Memphis , Tenn. 38108, dimensions: 4.5 cm Width×4.5 cm Long×0.92 mm thick. This material was selected on the basis of a high absorption of water and being made of natural components offering less possibility for contaminating insulin.

### Material # 2

Material: Non-woven polyester fiber blend, supplier model no.: M-261420025, Microdon-Web, supplied by: 3M Co., 3M Center, Bldg. 275-03E-10, PO Box 33275, St Paul, Minn. 55133, dimensions: 4.5 cm Width×4.5 cm Long×5 mm thick. This material was selected on the basis that it absorbs water at approx. 2 mls/sq. inch.

### Material # 3

Material: Polyproylene Non Woven fabric, supplier model no.: 3M Cotran 9729, supplied by: 3M Co., 3M Center, Bldg. 275-03E-10, PO Box 33275, St Paul, Minn. 55133, dimensions: 4.5 cm Width×4.5 cm Long×0.33 mm thick. This material was selected on the basis of having a moderate absorption of water, but that adheres the active pharmaceutical ingredient (API) as well.

### Experimental Pre-Treatment

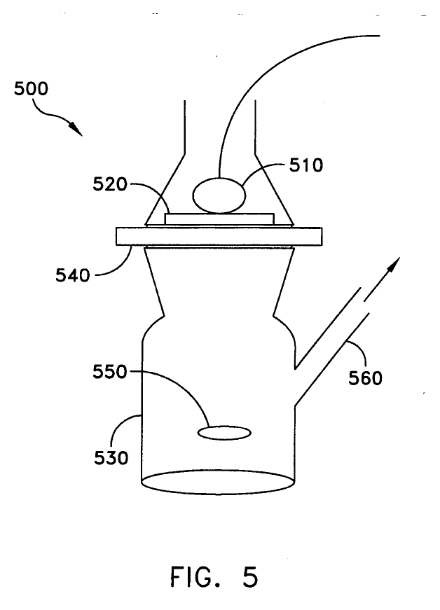

All three test materials were cut into a square shape of 1.75 inches×1.75 inches. The test square was attached to a Franz Diffusion cell, with a single element transducer placed directly above the absorbent square as is shown in FIG. 5. Franz Diffusion cell 500 of FIG. 5 includes an ultrasonic transducer 510, test pad 520, collection flask 530 and seal 540. A hydrophone 550 is positioned in the flask 530. Flask 530 also includes a sampling port 560.

Ultrasonic transducer 510 was set to generate an ultrasonic transmission of 20 KHz at 125 mW per sq. cm intensity. The absorbent square was treated for 60 continuous minutes and then removed and placed in a polybag, that was then heat sealed by an electric sealer device. The Bags were stored in a desiccant chamber until tested for ultrasonic attenuation.

Each absorbent material pad 520 was attached directly to transducer 510. Each absorbent pad was positioned 10 cm away from hydrophone 550 within flask 530, which contained distilled water at ambient temperature. Transducer 510 was a single element transducer set at varying ultrasonic frequency levels maintained at a standard intensity of 125 mW/cm².

The hydrophone's intensity output was read on an oscilloscope with the first measurement being made with no absorbent pad on the flask, to provide a control of the ultrasonic intensity without material interference. A second attenuation reading was then taken when the material pad was placed between the flask and the ultrasonic transducer. Two types of attenuation measurements were made. The first was the attenuation measured at 20 kHz at 10 cm between the sample and the hydrophone. The second was the attenuation measured at 40 kHz at 10 cm between the sample and the hydrophone. The results are expressed in Table-1 as a percentage compared with the control measure in the absence of the material sample.

TABLE 1

| | | MATERIAL-1 | | MATERIAL-2 | |
|---|---|---|---|---|---|
| MEASURE | MATERIAL-1 | FREEZE DRIED | MATERIAL-2 | FREEZE DRIED | MATERIAL-3 |
| ATTENUATION I | 44.9% | 49.5% | 59.4% | 37.1% | 63.9% |
| (20 kHz) | | | | | |
| ATTENUATION II | 25.4% | 41.9% | 48.5% | 43.6% | 50.5% |
| (40 kHz) | | | | | |

From these measurements, it may be seen that Material-1 (not freeze dried) and Material-2 (freeze-dried) show the smallest attenuation at 20 kHz pre-treatment. Material-3 crumbled when freeze-dried and did not maintain its integrity in a pad form. In general, the lower the attenuation is, the greater the effectiveness of ultrasound transduction through the material is. Hence, as seen from Table-1, with a 20 kHz frequency signal, on a comparative basis, freeze drying resulted in a decrease in attenuation for Material-2 and an increase in attenuation for Material-1. Hence Material-2 is better applicable to be employed as absorbent pad than Material-1.

Due to the intrinsic microstructure of certain materials, freeze drying can result in removal of air pockets within the material, which results in less or no resistance to ultrasound transduction through the material (e.g., material-2). Alternatively in case of certain materials, freeze drying may not have a significant effect on the microstructure, or actually result in an increase of the air pockets that ultimately results in increased resistance to ultrasound transduction and therefore increased attenuation. This may also be desirable in certain circumstances.

Tables 2 and 3 illustrate other experimental data.

TABLE 2
The Percentages of voltage ratio for different materials
measured @ 20 KHz

| Distance | Vi mV | Matl-1 mV | Matl- | Matl- | Matl- | | | Matl-2 | Matl-2 | Matl- | Matl-3 |
|---|---|---|---|---|---|---|---|---|---|---|---|
| cm | p-p | p-p | 1% | 1FD | 2FD % | Matl-2 | Matl-2% | FD | FD % | 3FD | FD % |
| 2 | 64 | 29 | 45.3 | 31 | 48.4 | 17 | 26.6 | 40 | 62.5 | 16 | 25.0 |
| 4 | 44 | 25 | 56.8 | 20 | 45.5 | 19 | 43.2 | 24 | 54.5 | 19 | 43.2 |
| 6 | 36 | 23 | 63.9 | 20 | 55.6 | 18 | 50.0 | 26 | 72.2 | 16 | 44.4 |
| 8 | 32 | 19 | 59.4 | 17 | 53.1 | 12 | 37.5 | 24 | 75.0 | 11 | 34.4 |
| 10 | 24 | 12 | 50.0 | 12 | 50.0 | 11 | 45.8 | 12 | 50.0 | 8 | 33.3 |

| | | | | | |
|---|---|---|---|---|---|
| The | 55.1 | 50.5 | 40.6 | 62.9 | 36.1 |
| average | | | | | |

TABLE 3

| Freq | Vo(mV | | Matl- | Matl-1 | Matl-1 | Matl-2 | Matl-2 | Matl-3 | Matl-3 | | Matl-3/ |
|---|---|---|---|---|---|---|---|---|---|---|---|
| (kHz) | p-p) | Matl-1 | 1/Vo | FD | FD/Vo | FD | FD/Vo | FD | FD/Vo | Matl-3 | Vo |
| 15 | 29 | 23 | 79.3 | 18 | 62.1 | 20 | 69.0 | 17 | 58.6 | 17 | 58.6 |
| 15.5 | 29 | 23 | 79.3 | 17 | 58.6 | 19 | 65.5 | 16 | 55.2 | 15 | 51.7 |
| 16 | 32 | 23 | 71.9 | 18 | 56.3 | 17 | 53.1 | 16 | 50.0 | 15 | 46.9 |
| 16.5 | 37 | 24 | 64.9 | 19 | 51.4 | 17 | 45.9 | 16 | 43.2 | 17 | 45.9 |
| 17 | 36 | 25 | 69.4 | 19 | 52.8 | 18 | 50.0 | 15 | 41.7 | 15 | 41.7 |
| 17.5 | 37 | 27 | 73.0 | 22 | 59.5 | 18 | 48.6 | 17 | 45.9 | 19 | 51.4 |
| 18 | 39 | 29 | 74.4 | 25 | 64.1 | 22 | 56.4 | 19 | 48.7 | 21 | 53.8 |
| 18.5 | 40 | 33 | 82.5 | 29 | 72.5 | 25 | 62.5 | 23 | 57.5 | 23 | 57.5 |
| 19 | 40 | 35 | 87.5 | 32 | 80.0 | 28 | 70.0 | 25 | 62.5 | 25 | 62.5 |
| 19.5 | 40 | 32 | 80.0 | 30 | 75.0 | 29 | 72.5 | 29 | 72.5 | 23 | 57.5 |
| 20 | 44 | 32 | 72.7 | 28 | 63.6 | 22 | 50.0 | 31 | 70.5 | 21 | 47.7 |
| 20.5 | 47 | 31 | 66.0 | 23 | 48.9 | 20 | 42.6 | 26 | 55.3 | 20 | 42.6 |
| 21 | 49 | 35 | 71.4 | 23 | 46.9 | 19 | 38.8 | 21 | 42.9 | 22 | 44.9 |
| 21.5 | 54 | 35 | 64.8 | 24 | 44.4 | 19 | 35.2 | 22 | 40.7 | 23 | 42.6 |
| 22 | 59 | 37 | 62.7 | 26 | 44.1 | 22 | 37.3 | 28 | 47.5 | 24 | 40.7 |
| 22.5 | 65 | 39 | 60.0 | 28 | 43.1 | 25 | 38.5 | 29 | 44.6 | 25 | 38.5 |
| 23 | 69 | 41 | 59.4 | 31 | 44.9 | 22 | 31.9 | 30 | 43.5 | 25 | 36.2 |
| 23.5 | 74 | 43 | 58.1 | 37 | 50.0 | 25 | 33.8 | 36 | 48.6 | 30 | 40.5 |
| 24 | 78 | 53 | 67.9 | 43 | 55.1 | 30 | 38.5 | 48 | 61.5 | 35 | 44.9 |
| 24.5 | 83 | 67 | 80.7 | 49 | 59.0 | 37 | 44.6 | 56 | 67.5 | 47 | 56.6 |
| 25 | 84 | 73 | 86.9 | 54 | 64.3 | 40 | 47.6 | 58 | 69.0 | 53 | 63.1 |
| 25.5 | 76 | 67 | 88.2 | 53 | 69.7 | 39 | 51.3 | 50 | 65.8 | 50 | 65.8 |
| 26 | 66 | 57 | 86.4 | 42 | 63.6 | 40 | 60.6 | 42 | 63.6 | 45 | 68.2 |
| 26.5 | 64 | 53 | 82.8 | 39 | 60.9 | 36 | 56.3 | 40 | 62.5 | 41 | 64.1 |
| 27 | 63 | 49 | 77.8 | 37 | 58.7 | 33 | 52.4 | 43 | 68.3 | 35 | 55.6 |
| 27.5 | 62 | 47 | 75.8 | 36 | 58.1 | 36 | 58.1 | 40 | 64.5 | 33 | 53.2 |
| 30 | 54 | 46 | 85.2 | 34 | 63.0 | 25 | 46.3 | 39 | 72.2 | 32 | 59.3 |
| 40 | 35 | 28 | 80.0 | 20 | 57.1 | 14 | 40.0 | 19 | 54.3 | 17 | 48.6 |

| | | | | | |
|---|---|---|---|---|---|
| Average | 74.6 | 58.1 | 49.9 | 56.4 | 51.4 |

Tables 2 and 3 illustrate raw data for experimentation performed to test Material 1, Material 2, and Material 3 for % attenuation to different frequencies using ultrasound transduction with and without freeze drying (FD). The highlighted row for frequency setting at 20 kHz shows the % attenuation of the three materials with and without freeze drying (FD)

Thus, it is demonstrated that pre-treating the absorbent material with ultrasound changes the absorbent material's ultrasound attenuation. It should be understood while it is surmised this results from driving moisture and air from the absorbent material, as well as other mechanisms described herein, this is not a limiting factor of the present invention. Further, the pre-treatment may occur prior to positioning it within a patch, or thereafter. And, the patch may be loaded with the substance to be delivered prior to positioning it within a patch, or thereafter. Thereafter, the patch is sealed for delivery to a user.

It will be apparent to those skilled in the art that modifications and variations may be made in the apparatus and process of the present invention without departing from the spirit or scope of the invention. It is intended that the present invention cover the modification and variations of this invention provided they come within the scope of the appended claims and their equivalents.

What is claimed is:

1. A method for preparing a patch containing at least one substance to be delivered into a user, said method comprising:
providing an absorbent material;
insonifying said absorbent material;
introducing the at least one substance into the insonified absorbent material;
placing the absorbent material inside the patch; and,
sealing the substance holding insonified absorbent material containing patch.

2. The method of claim 1, wherein the substance is delivered to the user dependently upon the patch being affixed within a functional proximity of the user and being ultrasonically activated, wherein the sonicating and ultrasonic activation use at least one substantially common frequency.

3. The method of claim 2, wherein the sonicating and ultrasonic activation use at least one substantially common intensity.

4. A method for preparing a patch containing at least one substance to be delivered into a user, said method comprising:
providing an absorbent material;
sonicating said absorbent material;
introducing the at least one substance into the sonicated material;
placing the sonicated material inside the patch; and,
sealing the substance holding sonicated material containing patch.

5. A method for preparing a patch containing at least one substance to be delivered into a user, said method comprising:
providing an absorbent material;
freeze drying said absorbent material;
introducing the at least one substance into the freeze dried material;
placing the freeze dried material inside the patch; and,
sealing the substance holding freeze dried material containing patch.

6. The method of claim 5, further comprising chemically treating said absorbent material prior to freeze drying.

7. The method of claim 5, further comprising introducing a salt into said absorbent material prior to freeze drying.

8. The method of claim 5, wherein said chemically treating comprises soaking said absorbent material in a salt solution.

9. The method of claim 8, wherein said salt solution is a 0.9% NaCl solution.

10. A substance delivery patch comprising:
a container; and,
a substance holding absorbent material in said container;
wherein, said substance and absorbent material are cross-linked.

11. The patch of claim 10, wherein said absorbent material is pre-treated to facilitate said cross-linking.

12. The patch of claim 10, wherein said absorbent material is insonified.

13. The patch of claim 10, wherein said absorbent material is freeze-dried.

14. The patch of claim 10, wherein said absorbent pad is washed.

15. The patch of claim 10, wherein said absorbent material comprises a salt distinct from said substance.

16. The patch of claim 10, further comprising an adhesive on said container for securing said container to a user, wherein said adhesive and substance are isolated from one another.
